(19) United States
(12) Patent Application Publication
(10) Pub. No.: US 2007/0134310 A1
(43) Pub. Date: Jun. 14, 2007

(54) Transdermal risperidone delivery system

(76) Inventors: Diane E. Nedberge, Los Altos, CA (US); Robert M. Gale, Los Altos, CA (US); Nieves M. Crisologo, Sunnyvale, CA (US); Jianye Wen, Palo Alto, CA (US); Delphine C. Imbert, Cupertino, CA (US); Allison Luciano, Lebanon, NJ (US); Eric N. Silverberg, Summit, NJ (US); Paul B. Foreman, Somerville, NJ (US); Johan H. Geerke, Los Altos, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/525,976

(22) Filed: Sep. 22, 2006

Related U.S. Application Data
(60) Provisional application No. 60/720,212, filed on Sep. 23, 2005.

(51) Int. Cl. A61K 9/70; A61K 31/519

(52) U.S. Cl. 424/449; 514/259.41

(57) ABSTRACT

A system for transdermal delivery of risperidone to an individual. The system has a high risperidone loading with suitable permeation enhancers to effect therapeutic flux rate. Acrylate polymeric reservoir with the high risperidone and permeation enhancers dissolved therein provides desirable adhesive characteristics and effective transdermal therapeutic properties.

### CROSS REFERENCE TO RELATED U.S. APPLICATION DATA

The present application is derived from and claims priority to provisional application U.S. Ser. No. 60/720,212, filed Sep. 23, 2005, which is herein incorporated by reference in its entirety.

### TECHNICAL FIELD

This invention relates to a medical patch for transdermal administration of risperidone and to a method of treating a subject by administering risperidone thereto with a medical patch. In particular, the invention relates to transdermal systems for administration of risperidone with adhesive system having high enhancer tolerance when used in transdermal drug delivery.

### BACKGROUND

Transdermal devices for the delivery of biologically active agents have been used for maintaining health and therapeutically treating a wide variety of ailments. For example, analgesics, steroids, etc., have been delivered with such devices. Such transdermal devices include patches in which a biologically active agent is delivered to the body tissue passively without use of an additional energy source. Many such devices have been described, for example, in U.S. Pat. Nos. 3,598,122, 3,598,123, 4,379,454, 4,286,592, 4,314,557, 4,568,343, and U.S. Application No. 2003002682, all of which are incorporated herein by reference.

A transdermal patch is typically a small adhesive bandage that contains the drug to be delivered. A simple type of such transdermal patches is an adhesive monolith including a drug-containing reservoir disposed on a backing. The reservoir is typically formed from a pharmaceutically acceptable pressure sensitive adhesive. In some cases, the reservoir can be formed from a non-adhesive material, the skin-contacting surface of which is provided with a thin layer of a suitable adhesive. The rate at which the drug is administered to the patient from these patches can vary due to normal person-to-person and skin site-to-skin site variations in the permeability of skin to the drug.

Sometimes patches can be multilaminate or can include a liquid reservoir layer in the patches. A drug release-rate controlling membrane can be disposed between the drug reservoir and the skin-contacting adhesive. This membrane, by decreasing the release rate of drug from the patch, serves to reduce the effects of variations in skin permeability.

Although the transdermal delivery of therapeutic agents has been the subject of intense research and development for over 30 years, only a relatively small number of drug molecules are suitable for transdermal delivery due to the fact that human skin is an excellent barrier. Various techniques have been explored to enhance the permeation of drug molecules that are not otherwise suitable for transdermal delivery. Of these techniques, chemical enhancement is the most established and is currently employed commercially. Pressure sensitive adhesives, such as acrylic adhesives, are used in most transdermal drug delivery devices as a means of providing intimate contact between the drug delivery device and the skin. The use of drugs and permeation enhancers (“enhancers”), especially at high concentrations, usually has a significant impact on the properties of pressure sensitive adhesives, such as cohesive strength, adhesive flow, tackiness and adhesion strength. Therefore, pressure sensitive adhesives have to be designed in a way that they can provide the needed performance in the presence of enhancer.

Risperidone (RISPERDAL® from Janssen Pharmaceutica Products) has been used for the management of psychotic symptoms associated with schizophrenia. Risperidone is chemically named 3-[2-[4-(6-fluoro-1,2-benzisoxazol-3-yl)-1-piperidinyl]ethyl]-6,7,8,9-tetrahydro-2-methyl-4H-pyrido [1,2-a]pyrimidin-4one. The preparation and pharmacological activity of risperidone are described in U.S. Pat. No. 4,804,663. It is used for producing an antipsychotic effect or alleviating behavioral disturbances associated with neurodegenerative disorders, such as schizophrenia and bipolar disorder. It is occasionally used to treat severe behavioral disorders in children and teenagers with autistic disorders. Risperidone is taken once or twice per day, by mouth. The dose is in the form of a tablet, a liquid, or an orally disintegrating tablet. See, e.g., Physicians Desk Reference, 57^{th} Edition, 2003, pages 1786-1790. It has been reported that for producing antipsychotic effect in a patient the daily dose is about 2 to 8 mg; for alleviation of behavioral disturbances associated with neurodegenerative disorders the daily dose is less. Internet publication “Risperidone: Schizophrenia Management Plan”, by Phillip W. Long in Internet Mental Health (www.mentalhealth.com) © 1995-2003, reported that risperidone is an antipsychotic drug useful in treating psychotic symptoms (as in schizophrenia) and that 6 mg daily dose (3 mg b.i.d.) had been proven to produce optimal therapeutic results. Transdermal administration has been described in patent document EP0879051, corresponding to WO96/31201. All such publications are incorporated by reference herein.

However, it is not easy to deliver an adequate amount of risperidone for effective treatment of neurological disorder such as schizophrenia, particularly sustained delivery over a period of time that is convenient to use, especially for individuals that may need assistance to receive medication orally or via injection. For the purpose of producing an antipsychotic effect in a patient the total daily dose of risperidone ranges from about 2 to about 8 mg. Thus far, there is still no transdermal risperidone delivery system of a convenient size that is applicable on a patient by a patient over a period of days and that has been shown to deliver a flux adequate for therapeutic effect. There continues to be a need for improved delivery of risperidone, especially sustained delivery over a period of time.

### SUMMARY

The present invention provides a method and a device for transdermal delivery of risperidone for therapeutic effects on neurological disorder such as schizophrenia and/or bipolar disorder, especially delivery of risperidone to a subject in need thereof through skin or other body surface that is accessible from exterior without using endoscopic devices. A patient can wear the device over an extended period of time. The transdermal delivery of this drug may result in lower adverse events (i.e. orthostatic hypotension) than seen with oral delivery. Further, a transdermal patch will allow a more steady sustained delivery than doses taken orally at time intervals hours apart. The transdermal form of the drug could allow use in the patient population that cannot take oral medication. This invention allows for the transdermal delivery of a therapeutic dose of risperidone (2 to 6 mg per day) from a thin, flexible, user-friendly patch between, e.g., 20 and 40 cm² in size. It also provides us with a method to load enough risperidone (preferably completely dissolved) into the drug reservoir of the transdermal patch that can be applied to a patient for an extensive period of time, such as 3, 7 days, or even longer. Patches that can be used for such extensive periods of time would increase patient compliance and will be less burdensome to care givers.

In one aspect, the present invention provides a system for transdermal delivery of risperidone. In another aspect, the present invention to provide a transdermal risperidone delivery system with improved enhancer loading, little or no cold flow, with adequate rheological and adhesive properties. The preferred acrylate proadhesive has a high functionality (e.g., acid and hydroxyl functional groups) for increasing hydrophilic and polar functionality. The increased loading of the present invention can allow for 7-day delivery at a reasonable adhesive thickness.

In a preferred mode, in a reservoir, an acrylate matrix material that is originally too stiff for pressure sensitive adhesive properties before incorporation of drug and permeation enhancers is used. It has been discovered that by increasing the glass transition temperature of the acrylate polymer using the ratio of soft monomer and hard monomer, it is possible to load enhancer concentrations into the polymer at a high weight percent to obtain a formulation and still achieve desirable adhesive characteristics.

It is possible to load drug and/or enhancer into the polymer composition to a high concentration, e.g., at greater than 20 dry weight %, greater than 30 dry weight % (or solids wt %), even up to 40-50 wt %, and still provide adequate adhesion and Theological characteristics for pressure sensitive adhesive (PSA) application. With sufficient loadings of permeation enhancers in such formulations, sustained high rates of drug delivery can be achieved. With adequate adhesive properties, the resulting reservoir with sufficient drug loading and permeation enhancers can be used to achieve effective therapeutic results. In certain embodiments with high loading, prior to incorporation of drug and other ingredients, the polymeric materials are not suitable PSAs “as is” because of the stiffness of the polymer and insufficient adhesiveness or tackiness. These polymeric materials become adhesive and have the desired PSA characteristics after incorporating drug, permeation enhancer and optionally other ingredients in suitable quantities. Such polymeric materials, which are not suitable as a PSA as is (prior to incorporation of drugs and ingredients) but will have the desired PSA characteristics after incorporating drugs and/or other ingredients, can be called “proadhesive” herein.

### BRIEF DESCRIPTION OF THE DRAWINGS

FIG. 1 illustrates a cross-section through a schematic, perspective view of one embodiment of a transdermal therapeutic system according to the present invention.

FIG. 2 illustrates a cross-section view through another embodiment of a transdermal therapeutic system of this invention.

FIG. 3 is a graph showing the flux data of transdermal risperidone delivery using systems of the present invention.

### DETAILED DESCRIPTION

The present invention relates to transdermal delivery of risperidone or a salt thereof, especially the uncharged base form of risperidone, with the help of permeation enhancers for loading adequate amount of risperidone.

A suitable transdermal delivery patch according to the present invention can deliver risperidone through about 5-100 cm², and preferably about 10-50 cm², more preferably about 20 cm² of intact skin over an extended period of time. For a 2 to 6 mg daily dose a transdermal risperidone flux in a range of 4-12.5 μg/cm²-hr for a system area of 20 cm² is needed. This range can be reduced to 2-6.5 μg/cm²-hr if the patch size is increased to 40 cm². The delivery of 6 mg per day from a 40 cm² twice-weekly patch (4 day delivery) requires a drug loading in excess of 9.6 wt % from a 5 mil drug reservoir if the drug depletion is limited to 50% during the 4 days of wearing. For effective therapy, the delivery of daily dose transdermal risperidone flux in a range of 2 or more μg/cm²-hr is needed. The wt % drug loading can be reduced if a thicker drug reservoir or a larger patch size is used. The risperidone can be included in the reservoir at an amount of about 1 to 20 wt %, preferably 4 to 20 wt %, preferably about 5 to 15 wt %. Thus, the reservoir can deliver the risperidone at a flux of greater than 2 μg/cm²-hr, preferably greater than 4 μg/cm²-hr.

Traditionally a transdermal drug delivery system was formulated with a pressure sensitive adhesive that has a glass transition temperature (T_{g}) in the range of −40° C. to −10° C. According to the present invention, a useful reservoir material is acrylate polymer. In one aspect of the present invention, one type of useful acrylate polymer for making a risperidone transdermal delivery patch is one that comprises, and preferably consists of 2-hydroxyethyl acrylate, vinyl acetate and 2-ethylhexyl acrylate. In one aspect of the present invention, a preferred starting acrylate polymeric material (which can be formulated into an adhesive material having high loading of pharmaceuticals and/or enhancers) has a glass transition temperature (T_{g}) in the range of −20° C. or higher, preferably −15° C. or higher, more preferably −15° C. to 0° C., and even more preferably −10° C. to 0° C.; creep compliance of about 7×10⁻⁵ cm²/dyn (at 3600 second) or below; and modulus G′ of about 8×10⁵ dyn/cm² or above. The polymeric material can be formulated into a transdermal reservoir matrix (including carrier structure) with a combined drug and/or enhancer concentration greater than 30 dry weight percent (wt %), or even greater than 40 dry weight percent. The resulting transdermal adhesive formulation with risperidone and preferably with enhancers will provide excellent adhesion with no cold flow, i.e., with no cold flow of an amount that is noticeable and would affect the normal use of the delivery system. By contrast, the proadhesive starting acrylate polymer has poor adhesive properties because the glass transition temperature is too high. Once plasticized in the transdermal formulation, the glass temperature drops into the pressure sensitive range, about −10 to −40° C., and the resulting creep compliance and storage modulus enables the achievement of good tack, with little or no cold flow. Creep compliance is an important parameter to evaluate cold flow behavior of a pressure sensitive adhesive (PSA). In a transdermal drug delivery system, if the creep compliance is large, the adhesive will have cold flow with time, i.e., the adhesive may lose its shape just because of the weight of the material in the device under gravity.

In describing the present invention, the following terms will be employed, and are intended to be defined as indicated below. As used in this specification and the appended claims, the singular forms “a,” “an” and “the” include plural references unless the text content clearly dictates otherwise.

As used herein, the term “transdermal” refers to the use of skin, mucosa, and/or other body surfaces as a portal for the administration of drugs by topical application of the drug thereto for passage into the systemic circulation.

“Biologically active agent” is to be construed in its broadest sense to mean any material that is intended to produce some biological, beneficial, therapeutic, or other intended effect, such as enhancing permeation, or relief of symptoms of neurological disorder. As used herein, the term “drug” refers to any material that is intended to produce some biological, beneficial, therapeutic, or other intended effect, such as relief of symptoms of neurological disorder, but not agents (such as permeation enhancers) the primary effect of which is to aid in the delivery of another biologically active agent such as the therapeutic agent transdermally.

As used herein, the term “therapeutically effective” refers to the amount of drug or the rate of drug administration needed to produce the desired therapeutic result. As used herein, the term “permeation enhancement” intends an increase in the permeability of skin to a drug in the presence of a permeation enhancer as compared to permeability of skin to the drug in the absence of a permeation enhancer. A “permeation-enhancing amount” of a permeation-enhancer is an amount of the permeation enhancer sufficient to increase the permeability of the body surface of the drug to deliver the drug at a therapeutically effective rate.

“Acrylate”, “polyacrylate” or “acrylic polymer”, when referring to a polymer for an adhesive or proadhesive, refers to polymer or copolymer of acrylic acid, ester(s) thereof, acrylamide, or acrylonitrile. Unless specified otherwise, it can be a homopolymer, copolymer, or a blend of homopolymers and/or copolymers.

As used in the present invention, “soft” monomers refer to the monomers that have a T_{g} of about −80 to −10° C. after polymerization into homopolymer; “hard” monomers refer to the monomers that have a T_{g} of about 0 to 250° C. after forming homopolymer; and “functional” monomers refer to the monomers that contain hydrogen bonding functional groups such as hydroxyl, carboxyl or amino groups (e.g., alcohols, carboxylic acid, or amines), these polar groups tend to increase the hydrophilicity of the acrylate polymer and increase polar drug solubility.

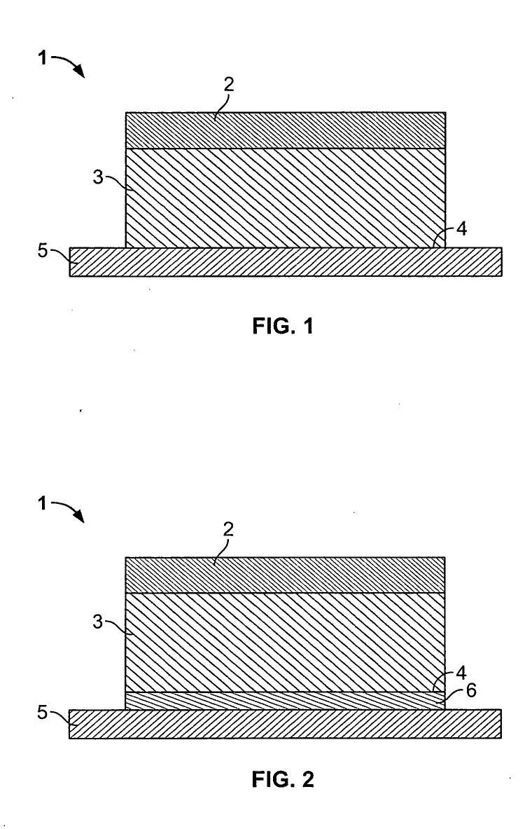

Exemplary transdermal drug delivery systems of the present invention are illustrated by the embodiments shown in FIGS. 1 and 2. As shown in FIGS. 1 and 2, an embodiment of the transdermal monolithic patch 1 according to this invention has a backing layer 2, a drug reservoir 3 disposed on the backing layer 2, and a peelable protective layer 5. In the reservoir 3, which can be a layer, at least the skin-contacting surface 4 is an adhesive. The reservoir is a matrix (carrier) that is suitable for carrying the pharmaceutical agent (or drug) for transdermal delivery. Preferably, the whole matrix, with drugs and other optional ingredients, is a material that has the desired adhesive properties. The reservoir 3 can be either a single phase polymeric composition or a multiple phase polymeric composition. In a single phase polymeric composition the drug and all other components are present at concentrations no greater than, and preferably less than, their saturation concentrations in the reservoir 3. This produces a composition in which all components are dissolved in the matrix. The reservoir 3 is formed using a pharmaceutically acceptable polymeric material that can provide acceptable adhesion for application to the body surface. In a multiple phase polymeric composition, at least one component, for example, a therapeutic drug, is present in amount more than the saturation concentration. In some embodiments, more than one component, e.g., a drug and a permeation enhancer, is present in amounts above saturation concentration. In the embodiment shown in FIG. 1, the adhesive acts as the reservoir and includes a drug.

In the embodiment shown in FIG. 2, the reservoir 3 is formed from a material that does not have adequate adhesive properties if without drug or permeation enhancer. In this embodiment of a monolithic patch 1, the skin-contacting surface of the reservoir 4 may be formulated with a thin adhesive coating 6. The reservoir 3 may be a single phase polymeric composition or a multiple phase polymeric composition as described earlier, except that it may not contain an adhesive with adequate adhesive bonding property for skin. The adhesive coating can contain the drug and permeation enhancer, as well as other ingredients.

The drug reservoir 3 is disposed on the backing layer 2. At least the skin-contacting surface of the reservoir is adhesive. As mentioned, the skin-contacting surface can have the structure of a layer of adhesive. The reservoir 3 may be formed from drug (or biological active agent) reservoir materials as known in the art. For example, the drug reservoir is formed from a polymeric material in which the drug has reasonable solubility for the drug to be delivered within the desired range, such as, a polyurethane, ethylene/vinyl acetate copolymer (EVA), acrylate, styrenic block copolymer, and the like. In preferred embodiments, the reservoir 3 is formed from a pharmaceutically acceptable adhesive or proadhesive, preferably acrylate copolymer-based, as described in greater detail below. The drug reservoir or the matrix layer can have a thickness of about 1-10 mils (0.025-0.25 mm), preferably about 2-5 mils (0.05-0.12 mm), more preferably about 2-3 mils (0.05-0.075 mm).

Preferred materials for making the adhesive reservoir or adhesive coating, and for making proadhesives according to the present invention include acrylates, which can be a copolymer of various monomers ((i) “soft” monomer, (ii) “hard” monomer, and optionally (iii) “functional” monomer) or blends including such copolymers. The acrylates (acrylic polymers) can be composed of a copolymer (e.g., a terpolymer, i.e., made with three monomers; or a tetrapolymer, i.e., made with four monomers) including at least two or more exemplary components selected from the group including acrylic acids, alkyl acrylates, methacrylates, copolymerizable secondary monomers or monomers with functional groups. Functional monomers are often used to adjust drug solubility, polymer cohesive strength, or polymer hydrophilicity. Examples of functional monomers are acids, e.g., acrylic acid, methacrylic acid and hydroxy-containing monomers such as hydroxyethyl acrylate, hydroxypropyl acrylate, acrylamides or methacrylamides that contain amino group and amino alcohols with amino group protected. Functional groups, such as acid and hydroxyl groups can also help to increase the solubility of basic ingredients (e.g., drugs) in the polymeric material. Additional useful “soft” and “hard” monomers include, but are not limited to, methoxyethyl acrylate, ethyl acrylate, butyl acrylate, butyl methacrylate, hexyl acrylate, hexyl methacrylate, 2-ethylbutyl acrylate, 2-ethylbutyl methacrylate, isooctyl acrylate, isooctyl methacrylate, 2-ethylhexyl acrylate, 2-ethylhexyl methacrylate, decyl acrylate, decyl methacrylate, dodecyl acrylate, dodecyl methacrylate, tridecyl acrylate, tridecyl methacrylate, acrylonitrile, methoxyethyl acrylate, methoxyethyl methacrylate, and the like. Additional examples of acrylic adhesive monomers suitable in the practice of the invention are described in Satas, “Acrylic Adhesives,” Handbook of pressure-Sensitive Adhesive Technology, 2nd ed., pp. 396-456 (D. Satas, ed.), Van Nostrand Reinhold, New York (1989). Examples of acrylic adhesives are commercially available from National Starch and Chemical Company, Bridgewater, N.J.

The acrylate polymers can include cross-linked and non-cross-linked polymers. The polymers can be cross-linked by known methods to provide the desired polymers. However, cross-linking is hard to control and may result in polymeric materials that are too stiff or too soft. According to the present invention, it is preferred that the polymeric material for incorporation of drugs and other ingredients to be polymers without crosslinking and no cross-linking agent is used in forming the polymeric material. It is further preferred that the monomers do not self cross-link during polymerization. In the present invention, it was found that, instead of cross-linking to form a matrix adhesive with desired PSA properties for incorporating drugs and enhancers, good control of the PSA properties can be achieved by selecting polymeric materials, and in one aspect related to proadhesive, selecting materials that are too stiff prior to incorporation of drugs and other ingredients and subsequently incorporating such drugs and ingredients.

It has been found that, in the case of proadhesive, in a preferred embodiment, an acrylate polymer composition with a creep compliance (J) of 7×10⁻⁵ cm²/dyn or below and elastic modulus G′ of 8×10⁵ dyn/cm² or above, although too stiff as a PSA as is, after formulating with drug or enhancer or a combination thereof at a relative high concentration will achieve the desirable adhesive properties. The plasticizing or tackifying effect of the drug(s) and/or other ingredients on the polymeric material provides a means to achieve the desired adhesive properties in the reservoir.

Acrylate polymers, when the main monomer of which has the general formula CH₂═CH—COOR, are particularly useful as proadhesives. Typical main monomers are normally alkyl acrylates of 4 to 1 carbon atoms, preferably 4-10 carbons. Useful alkyl acrylates include ethyl acrylate, butyl acrylate, amyl acrylate, hexyl acrylate, 2-ethylhexyl acrylate, octyl acrylate, isooctyl acrylate, decyl acrylate, dodecyl acrylates, with 2-ethylhexyl acrylate, butyl acrylate, and iso-octyl acrylate being preferred. Such “soft” monomers if polymerized into homopolymer generally have a T_{g} of less than about 0° C., preferably about −10° C. to −80° C., preferably about —20° C. to −80° C. Preferably, they are present in an amount of about 10 to 70 wt % (i.e., dry weight % or solids wt %), more preferably no more than about 60% by weight, more preferably no more than about 50 wt % of the total monomer weight and more preferably about 40 to 50 wt %. As used herein, when a monomer is said to be present in the acrylate polymer at a certain percentage, it is meant that the monomer has been polymerized in the acrylate polymer at that percentage of polymerization monomer ingredients.

“Hard” modifying monomers are mainly used to modify the adhesive properties, mainly glass transition temperature (e.g., to increase the T_{g} and to make the resulting polymer stiffer at room temperature), to meet various application requirements. A hard monomer, if polymerized into homopolymer, has a T_{g} of about 0 to 250° C., preferably about 20 to 250° C., more preferably in the range of about 30 to 150° C. (for convenience, this is referred to as the “homopolymer T_{g}” herein). The hard monomer component is present in an amount of about 10 wt % or more, preferably in the range of about 30 to 60 wt %, preferably about 35 to 60 wt %, more preferably about 40 to 60 wt %, even more preferably about 40 to 50 wt % in the polymerization. Examples of hard modifying monomers are methyl acrylate, vinyl acetate, methyl methacrylate, isobutyl methacrylate, vinyl pyrrolidone, substituted acrylamides or methacrylamides. Homopolymers of these monomers generally have higher glass transition temperature than homopolymers of the soft monomers.

Certain nitrogen containing monomers can be included in the polymerization to raise the T_{g}. These include N-substituted acrylamides or methacrylamides, e.g., N-vinyl pyrrolidone, N-vinyl caprolactam, N-tertiary octyl acrylamide (t-octyl acrylamide), dimethyl acrylamide, diacetone acrylamide, N-tertiary butyl acrylamide (t-butyl acrylamide and N-isopropyl acrylamide (i-propyl acrylamide). Further examples of monomers that can be used in polymerization to modify and raise the T_{g} of the polymer include cyanoethylacrylates, N-vinyl acetamide, N-vinyl formamide, glycidyl methacrylate and allyl glycidyl ether.

Functional monomers can be used to either provide needed functionality for solubilizing agents in the polyacrylate or improve cohesive properties. Examples of functional monomers are organic acids, e.g., acrylic acid, methacrylic acid, and hydroxyl-containing monomers such as hydroxyethyl acrylate. Preferred functional monomers when incorporated into the polymer result in acid groups, i.e., —COOH, hydroxyl groups, i.e., —OH, or amino groups in the polymer for affecting the solubility of basic agents such as basic drugs. Examples of hydroxy functional monomers include hydroxyethyl acrylate, hydroxypropyl acrylate, hydroxyethyl methacrylate and hydroxypropyl methacrylate. The hydroxyl groups can be primary, secondary or tertiary hydroxyl. In some cases, the acrylate polymer can includes at least one non-primary hydroxyl functional monomer component to provide orientation of the functional group in the polymer. Suitable non-primary hydroxyl functional monomers are secondary hydroxyl functional monomers such as hydroxypropyl acrylate. Useful carboxylic acid monomers to provide the functional group preferably contain from about 3 to about 6 carbon atoms and include, among others, acrylic acid, methacrylic acid, itaconic acid, and the like. Acrylic acid, methacrylic acid and mixtures thereof are particularly preferred as acids.

A functional monomer can also be a hard monomer, if its homopolymer has the high T_{g}. Such functional monomers that can also function as hard monomers include, e.g., hydroxyethyl acrylate, hydroxypropyl acrylate, acrylic acid, dimethylacrylamide, dimethylaminoethyl methacrylate, tert-butylaminoethyl methacrylate, methoxyethyl methacrylate, and the like.

The functional monomer(s) are preferably present in the acrylate polymer in an amount of about at least 5 wt %, preferably at least 10 wt %, preferably 10 to 40 wt %, more preferably about 10 to 30 wt %, more preferably about 10 to 20 wt %, even more preferably 10 to 15 wt %, even though some of the functional monomer(s) may be hard monomers. Examples of preferred functional monomer component include acrylic acid and hydroxyethyl acrylate, acrylamides or methacrylamides that contain amino group and amino alcohols with amino group protected. One of the applications of using functional monomers is to make a polar proadhesive having higher enhancer tolerance, in that, for example, the resulting PSA with the enhancers and/or drug will not phase separate or have excessive cold flow.

In certain embodiments, the hard monomer(s) that are not also functional monomer can constitute about 10 to 60 wt %, preferably about 40 to 60 wt % of the acrylate monomer, especially in cases in which no acidic functional hard monomer and less than about 20 wt % of hydroxyl functional hard monomer are included in the acrylate polymer. In other embodiments, the hard monomer(s) that are not also functional monomer can constitute about 5 to 15 wt %, e.g., about 10 wt % of the acrylate monomer, especially in cases in which a large amount (e.g., about 25 wt % or more) of functional hard monomer(s) are included, such as when more than about 5 wt % acidic hard functional monomers and 10 or more wt % (e.g., about 10-25 wt %) hydroxyl functional hard monomer(s) are included in the acrylate polymer.

In an embodiment, useful polyacrylates have at least about 10 wt %, preferably at least about 20 wt %, preferably at least about 30 wt % acrylic monomers having hydroxyl group, acid group, or a combination thereof. One example is a polyacrylate having about 30 wt % hydroxyl group containing (—OH) monomer and about 3 wt % acid containing (—COOH) monomer. Another contains about 26 wt % —OH monomer and about 6 wt % —COOH monomer. Another useful polar polyacrylate contains about 10 wt % —OH monomer. Yet another useful polar polyacrylate contains about 20 wt % —OH monomer. The preferred —OH monomer is hydroxyethyl acrylate and hyderoxylpropyl acrylate. The preferred —COOH monomer is acrylic acid. Proadhesives were made with such functional amounts.

Below is a table showing the T_{g}'s of exemplary soft and hard homopolymers the monomers of which are useful for making adhesive and proadhesive of the present invention. Some of the monomers (e.g., acrylic acid, hydroxyethyl acrylate) are also functional monomers.

| poly(hydroxyethyl acrylate) (hard/functional monomer) | about 100° | C. |
|---|---|---|
| poly(acrylic acid) (hard/functional monomer) | 106° | C. |
| poly(vinyl acetate) (hard monomer) | 30° | C. |
| poly(ethylhexyl acrylate) (soft monomer) | −70° | C. |
| poly(isopropyl acrylate) (soft monomer) | −8° | C. |
| poly(n-propyl acrylate) (soft monomer) | −52° | C. |
| poly(isobutyl acrylate) (soft monomer) | −40° | C. |
| poly(n-butyl acrylate) (soft monomer) | −54° | C. |
| poly(n-octyl acrylate) (soft monomer) | −80° | C. |

It has been found that the soft monomers 2-ethylhexyl acrylate and butyl acrylate are especially suitable to polymerize with functional monomers hydroxyethyl acrylate or acrylic acid either alone or in combination to form the acrylate polymer of the present invention. Further, the hard monomer vinyl acetate has been found to be very useful to polymerize with the soft monomers 2-ethylhexyl acrylate and butyl acrylate, either alone or in combination to form the proadhesive. Thus, the acrylate proadhesive polymer of the present invention is especially suitable to be made from 2-ethylhexyl acrylate or butyl acrylate copolymerized with hydroxyethyl acrylate, acrylic acid, or vinyl acetate, either alone or in combination. Another preferred hard monomer is t-octyl acrylamide, which can be used alone or in combination with other hard monomers such as acrylic acid and hydoxyethyl acrylate.

In an embodiment, the proadhesive is made by polymerizing monomers including about 30 to 75 wt % vinyl acetate, about 10-40 wt % hydroxyl functional monomer and about 10-70 wt % soft monomer such as 2-ethylhexyl acrylate or butyl acrylate. In a preferred embodiment, the proadhesive is made by polymerizing monomers including about 50 to 60 wt % vinyl acetate, about 10-20 wt % hydroxyethyl acrylate, and about 20-40 wt % 2-ethylhexyl acrylate. In some cases, no carboxyl (acid) group is used. Hydroxyethyl acrylate or hydroxypropyl acrylate can be used to provide hydroxyl functionality. For example, one embodiment is a proadhesive having about 50 wt % vinyl acetate, about 10 wt % hydroxyethyl acrylate, and about 40 wt % 2-ethylhexyl acrylate. As used herein, when a specific percentage is mentioned, it is contemplated there may be slight variations, e.g., of pius or minus 5% of the specific percentage (i.e., about 10 wt % may included 10 wt %±0.5wt %). One other embodiment is a proadhesive having about 60 wt % vinyl acetate, about 20 wt % hydroxyethyl acrylate, and about 20 wt % 2-ethylhexyl acrylate.

In another embodiment, the proadhesive is made by polymerizing monomers including both monomer with hydroxyl group and monomer with carboxyl group. For example, certain preferred monomer combination for polymerization include an alkyl acrylate, an acrylamide, a monomer with hydroxyl group and a monomer with carboxyl group, e.g., making a proadhesive by polymerizing butyl acrylate, 2-hydroxyethyl acrylate or 2 hydroxypropyl acrylate or hydroxypropyl methacrylate, t-octyl acrylamide, and acrylic acid. In an embodiment, greater than 3 wt % of a hydroxypropyl acrylate or hydroxylpropyl methacrylate is used in making the acrylate polymer.

In certain cases for making a proadhesive in which both monomers with hydroxyl groups and monomer with carboxyl groups are to be polymerized with a soft monomer, e.g., butyl acrylate, the monomer proportions in the polymerization includes about 55 to 65 wt % soft monomer (e.g., butyl acrylate), about 5 to 15 wt % t-octyl acrylamide, about 20 to 30 wt % hydroxyethyl or hydroxypropyl acrylate and about 5 to 10 wt % acid monomer such as acrylic acid. In one embodiment, the acrylate polymer includes about 59 wt % butyl acrylate, about 10 wt % t-octyl acrylamide, about 25 wt % hydroxypropyl acrylate and about 6 wt % acrylic acid. In another embodiment, the hydroxypropyl acrylate is replaced with hydroxyethyl acrylate.

It is desirable that with the incorporation of a large amount of permeation enhancers, the T_{g} of the resulting reservoir (with the drug, permeation enhancers and other ingredients) is such that the resulting reservoir would have good PSA properties for application to the body surface of an individual. Further, the resulting reservoir should not have cold flow that affects the normal application of the transdermal delivery. The acrylate polymer (or a blend of acrylate polymers) constitutes preferably about 40 wt % to 90 wt %, more preferably about 45 wt % to 80 wt % of the reservoir. It is possible to load drug and/or enhancer into the polymer composition to a high concentration, e.g., at or greater than about 20 dry weight %, at or greater than about 30 dry weight % (or solids wt %), even up to about 40 to 50 wt %, without adversely impacting the adhesion and rheological characteristics for pressure sensitive adhesive (PSA) application.

Preferred acrylate polymers or blends thereof provide the acrylic pressure sensitive properties in the delivery system glass transition temperature of about −10 to −40° C., preferably about −20 to −30° C. at application on a surface. The T_{g} of an acrylate polymer can be determined by differential scanning calorimetry (DSC) known in the art. Also, theoretical ways of calculating the T_{g} of acrylate polymers are also known. Thus, one having a sample of an acrylate polymer will be able to experimentally determine the T_{g}, for example, by DSC. One can also determine the monomer composition of the acrylate polymer and estimate theoretically the T_{g} by calculation. From the knowledge of the monomer composition of an acrylate polymer having drugs and enhancers, one can also make the acrylate polymer without the drug and enhancer and determine the T_{g}. According to the present invention, the acrylate materials, before dissolving the drug(s), permeation enhancers, etc., have T_{g}'s that are in the range of about −20 to 10° C., and have rheological properties that are not quite suitable for use directly as a PSA to skin because of the stiffness of the material. The acrylate polymers preferably have a molecular weight in a range of about 200,000 to 600,000. Molecular weight of acrylate polymers can be measured by gel permeation chromatography, which is known to those skilled in the art.

To control the physical characteristics of the acrylate polymer and the polymerization, it is preferred that monomers of molecular weight of below 500, even more preferably below 200 be used in the polymerization. Further, although optionally larger molecular weight monomers (linear macromonomers such as ELVACITE™ from ICI) can be used in the polymerization, it is preferred that they are not used. Thus, preferably no monomer of molecular weight (MW) above 5000, more preferably no monomer of MW above 2000, even more preferably no monomer of MW above 500, is to be included in the polymerization to form the acrylate polymer. The adhesives and proadhesives of the present invention can be formed without such macromonomers. Thus, in an aspect of the present invention, preferably, proadhesive polymers can be formed without macromonomers, or substantially without macromonomers, to have adhesive properties too stiff for PSA as is without incorporation of a large amount of permeation enhancers and drug. However, such proadhesives will become suitable for adhering to the skin as PSA in patch application after the appropriate amount of permeation enhancers and drug are dissolved therein.

However, if desired, in certain embodiments, optionally, the reservoir can include diluent materials capable of reducing quick tack, increasing viscosity, and/or toughening the reservoir structure, such as polybutylmethacrylate (ELVACITE, manufactured by ICI Acrylics, e.g., ELVACITE 1010, ELVACITE 1020, ELVACITE 20), polyvinylpyrrolidone, high molecular weight acrylates, i.e., acrylates having an average molecular weight of at least 500,000, and the like.

The acrylate polymers of the present invention can dissolve a large amount of permeation enhancer and allow the resulting drug and permeation enhancer-containing adhesive to have the desired adhesive and cohesive property without the drug or permeation enhancer separating out of the acrylate polymer matrix either as crystals or as oil. The resulting composition will be in the T_{g} and compliance range that it can be applied to a body surface without leaving an undesirable amount of residue material on the body surface upon removal of the device. The preferred acrylate polymer is not cross-linked. It is contemplated, however, that if desired, a nonsubstantial amount of cross-linking may be done, so long as it does not change substantially the T_{g}, creep compliance and elastic modulus of the acrylate polymer. It is found that higher T_{g} and higher molecular weight of the acrylate are important for the acrylate polymer tolerating high drug loading and enhancer loading. Since the measurement of the molecular weight of an acrylate polymer is difficult, precise or definite values are often not obtainable. More readily obtainable parameters that are related to molecular weight and drug and enhancer tolerance (i.e., solubility) are creep compliance and elastic modulus.

Enhancers typically behave as plasticizers to acrylate adhesives. The addition of an enhancer will result in a decrease in modulus as well as an increase in creep compliance, the effect of which is significant at high enhancer loading. A high loading of enhancers will also lower the T_{g} of the acrylate polymer. Thus, to achieve a proadhesive that is tolerant of high enhancer loading, other than increasing the T_{g} by using a higher ratio of hard monomer to soft monomer and the selection of suitable monomers, it is desirable to provide suitable higher molecular weight such that chain entanglement would help to achieve the desirable rheology. As a result, selecting a higher T_{g} and higher molecular weight for a proadhesive will increase the elastic modulus and decrease the creep compliance of the acrylate, making the proadhesive more enhancer tolerant. The measurement of the molecular weight of an acrylate polymer is often method-dependant and is strongly affected by polymer composition, since acrylate polymers discussed here are mostly copolymers, not homopolymers. More readily obtainable parameters that relate to molecular weight and drug and enhancer tolerance (i.e., solubility) are creep compliance and elastic modulus.

According to the present invention, especially useful polymeric materials for forming drug-containing PSA are acrylate polymers that, before the incorporation of drugs, enhancers, etc., and other ingredients for transdermal formation, have creep compliance (measured at 30° C. and 3600 second) of about 7×10⁻⁵ cm²/dyn or below and storage modulus G′ about 8×10⁵ dyn/cm² or above. Preferably the creep compliance is about 6×10⁻⁵ cm²/dyn to 2×10⁻⁶ cm²/dyn, more preferably about 5×10⁻⁵ cm²/dyn to 4×10⁻⁶ cm²/dyn. Preferably the storage modulus is about 8×10⁵ dyn/cm² to 5×10⁶ dyn/cm², more preferably about 9×10⁵ dyn/cm² to 3×10⁶ dyn/cm². Such creep compliance and modulus will render these acrylate polymers too stiff and unsuitable “as is” for dermal PSA applications. However, it was found that after formulating into a transdermal system with drugs, permeation enhancers, and the like, which produce plasticizing effect as well as tackifying effect, the acrylate polymers plasticized with permeation enhancers and/or drug would have a desirable storage modulus and creep compliance that are suitable for transdermal PSA applications. For example, the plasticized material would have a resulting creep compliance that is about 1×10⁻³ cm²/dyn or less, preferably more than about 7×10⁻⁵ cm²/dyn, preferably from about 7×10⁻⁵ cm²/dyn to 6×10⁻⁴ cm²/dyn, more preferably about 1×10⁻⁴ cm²/dyn to 6×10⁻⁴ cm²/dyn. The preferred storage modulus of the plasticized acrylate polymer is about 1×10⁵ dyn/cm² to 8×10⁵ dyn/cm², preferably about 1.2×10⁵ dyn/cm² to 6×10⁵ dyn/cm², more preferably about 1.4×10⁵ dyn/cm² to 5×10⁵ dyn/cm².

It was found that incorporating the proper selection of drug and other ingredients (such as permeation enhancer) and using the appropriate amounts thereof can change the T_{g}, storage modulus G′, and creep compliance sufficiently to result in an effective transdermal drug delivery system with the right adhesive properties for the desirable length of time, such as 24 hours, 3 day, or even 7 day application on a body surface. Such transdermal drug delivery systems will have little or no cold flow. As used herein, “little cold flow” means that any shape change of the device caused by cold flow is not noticeable by an average person on which the device is applied over the time of use. Particularly useful for forming adhesives incorporating an increased amount of beneficial agents (including drugs and permeation enhancers) over prior adhesives in transdermal drug delivery are the acrylic formulations containing a relatively lower percentage of soft monomers. It has been found that increasing the molecular weight increases the modulus of elasticity and decreases the polymer chain mobility via chain entanglements. Also, increasing hard monomer content increases the glass transition temperature.

It is contemplated that the reservoir 3 or the adhesive coating 6 can also be formed from other material that has pressure sensitive adhesives characteristics with the drug and permeation enhancers incorporated therein. Examples of reservoir material and pressure sensitive adhesives include, but are not limited to, acrylates, polysiloxanes, polyisobutylene (PIB), polyisoprene, polybutadiene, styrenic block polymers, and the like. Examples of styrenic block copolymer-based adhesives include, but are not limited to, styrene-isoprene-styrene block copolymer (SIS), styrene-butadiene-styrene copolymer (SBS), styrene-ethylenebutene-styrene copolymers (SEBS), and di-block analogs thereof. As mentioned, a preferred reservoir material is acrylate polymer.

As aforementioned, the reservoir 3 can include a single phase polymeric composition, free of undissolved components, containing an amount of the drug risperidone sufficient to induce and maintain the desired therapeutic effect in a human for at least three days. Other drugs can also be included in the risperidone-containing matrix.

As indicated in the above, in some embodiments, the reservoir or the adhesive may contain additional components such as, additives, permeation enhancers, stabilizers, dyes, diluents, plasticizer, tackifying agent, pigments, carriers, inert fillers, antioxidants, excipients, gelling agents, anti-irritants, vasoconstrictors and other materials as are generally known to the transdermal art. Typically, such materials are present below saturation concentration in the reservoir.

Permeation enhancers can be useful for increasing the skin permeability of the drug risperidone to achieve delivery at therapeutically effective rates. Such permeation enhancers can be applied to the skin by pretreatment or currently with the drug, for example, by incorporation in the reservoir. A permeation enhancer should have the ability to enhance the permeability of the skin for one, or more drugs or other biologically active agents. A useful permeation enhancer would enhance permeability of the desired drug or biologically active agent at a rate adequate to achieve therapeutic plasma concentrations from a reasonably sized patch (e.g., about 5 to 80 cm²). Examples of useful permeation enhancers include, but are not limited to, fatty acid esters of alcohols, including glycerin, such as capric, caprylic, dodecyl, oleic acids; fatty acid esters of isosorbide, sucrose, polyethylene glycol; caproyl lactylic acid; laureth-2; laureth-2 acetate; laureth-2 benzoate; laureth-3 carboxylic acid; laureth-4; laureth-5 carboxylic acid; oleth-2; glyceryl pyroglutamate oleate; glyceryl oleate; N-lauroyl sarcosine; N-myristoyl sarcosine; N-octyl-2-pyrrolidone; lauraminopropionic acid; polypropylene glycol-4-laureth-2; polypropylene glycol-4-laureth-5dimethy-1 lauramide; lauramide diethanolamine (DEA). Preferred enhancers include, but are not limited to, lauryl pyroglutamate (LP), glyceryl monolaurate (GML), glyceryl monocaprylate, glyceryl monocaprate, glyceryl monooleate (GMO), oleic acid, N-lauryl sarcosine, ethyl palmitate, laureth-2, laureth-4, and sorbitan monolaurate. Additional examples of suitable permeation enhancers are described, for example, in U.S. Pat. Nos.: 5,785,991; 5,843,468; 5,882,676; and 6,004,578.

In some embodiments, especially some in which the reservoir does not necessarily have adequate adhesive properties and a separate adhesive layer is used, a dissolution assistant can be incorporated in the reservoir to increase the concentration of the drug or biologically active ingredient within the reservoir layer. Surfactants and dissolution assistants can be used in combination to increase the delivery rate of risperidone. Permeation enhancers/acids that will improve drug solubility in the drug reservoir include: oleic acid, lactic acid, adipic acid, succinic acid, glutaric acid, sebacic acid, and hydroxycaprilic acid. Glacial acetic acid is also useful as a solubilization assistant. Permeation enhnacers can also act as solubilization assistants.

The permeation enhancers that are particularly useful in the transdermal delivery of risperidone include NLS: N-lauroyl sarcosine (fatty acid), OCP: octyl pyroglutamate (amide), IPP: isopropyl myristate (fatty ester), LL: lauryl lactate (fatty acid ester), OA: oleic acid (fatty acid), LRA: lauric acid (fatty acid), GMO: glycerol monooleate (fatty acid ester), GML: glycerol monolaurate (fatty acid ester), LTH: laureth-4 (fatty alcohol ether), OL: oleth-4(fatty alcohol ether), ETD: ethoxydiglycol (fatty acid ester), and LPY: lauryl pyrrolidone (amide), LAU: laureth-2 (fatty alcohol ether), and ISO: isosorbide (carbohydrate). In general, enhancers with solubility parameters lower than both that of the adhesive and that of the drug are effective in increasing risperidone flux through skin in vitro. The enhancement ratio (ER) is defined as (average risperidone transdermal flux from test formulation divided by average risperidone transdermal flux from control formulation).

In some embodiments, a large amount of permeation enhancer may be needed to aid the drug in transdermal delivery. The present invention is especially suitable for such transdermal delivery systems. In such cases, one or more permeation enhancers, alone or in combination, and which may act or include dissolution assistants, can constitute about 5 to 40% by weight, preferably about 10 to 35% by weight, and more preferably about 15 to 30% by weight solids of the resulting reservoir that has adequate pressure sensitive adhesive properties. As used herein, the term “combination” when refers to selection of two or more chemicals means the chemicals are selected together and not necessarily that they be chemically combined together in a reaction.

In certain embodiments, polyvinylpyrrolidone (PVP) can be incorporated into the acrylate polymer matrix to increase risperidone solubility and yet provide acceptable adhesive and cohesive properties for transdermal risperidone delivery. The incorporation of PVP results in an increase in modulus and decrease in creep compliance. PVP works particularly well with acrylate polymer adhesives that contain hydroxyl or acid functionalities, or both.

In the present invention, using the permeation enhancers suitable for enhancing solubility and flux of risperidone, optionally, no propylene glycol or eucalyptus oil need to be use to achieve the risperidone flux desired for effective therapy.

In some embodiments, a large amount permeation enhancer preferably is used to aid the transdermal delivery of risperidone. In such cases, one or more permeation enhancers, alone or in combination, and which may include dissolution assistants, can consititute about 10 to 40% by weight, preferably 15 to 40% by weight, preferably 15 to 30% by weight, preferably higher than 20% by weight solids of the matrix that has adequate pressure sensitive adhesive property. For effective delivery of risperidone, it has been found that a ratio of the amount (in wt %) of risperidone to the amount of permeation enhancer (or a plurality of enhancers) of 0.1 to 2.0 is preferred, in the range of 0.25 to 0.5 is more preferred. With the inclusion of the suitable permeation enhancers, preferably risperidone can be solubilized in the matrix of the drug reservoir to a concentration on solids of higher than 5 wt %, preferably from 5 to 20 wt % for multiple day delivery.

In certain embodiments, optionally, certain other plasticizer or tackifying agent is incorporated in the polyacrylate composition to improve the adhesive characteristics. Examples of suitable tackifying agents include, but are not limited to, aliphatic hydrocarbons; aromatic hydrocarbons; hydrogenated esters; polyterpenes; hydrogenated wood resins; tackifying resins such as ESCOREZ, aliphatic hydrocarbon resins made from cationic polymerization of petrochemical feedstocks or the thermal polymerization and subsequent hydrogenation of petrochemical feedstocks, rosin ester tackifiers, and the like; mineral oil and combinations thereof. The tackifying agent employed should be compatible with the polymer or blend of polymers. Other drugs that can be contained in the drug reservoir include, for example, those disclosed in U.S. Pat. No. 6,004,578. One skilled in the art will be able to incorporate such drugs based on the disclosure of the present invention.

As shown in FIGS. 1 and 2, the patch 1 can further includes a peelable protective layer 5. The protective layer 5 can be made of a polymeric material that may be optionally metallized. Examples of the polymeric materials include polyurethane, polyvinyl acetate, polyvinylidene chloride, polypropylene, polycarbonate, polystyrene, polyethylene, polyethylene terephthalate, polybutylene terephthalate, paper, and the like, and a combination thereof. In preferred embodiments, the protective layer includes a siliconized polyester sheet.

The backing layer 2 may be formed from any material suitable for making transdermal delivery patches, such as a breathable or occlusive material including fabric or sheet, made of polyvinyl acetate, polyvinylidene chloride, polyethylene, polyurethane, polyester, ethylene vinyl acetate (EVA), polyethylene terephthalate, polybutylene terephthalate, coated paper products, aluminum sheet and the like, or a combination thereof. In preferred embodiments, the backing layer includes low density polyethylene (LDPE) materials, medium density polyethylene (MDPE) materials or high density polyethylene (HDPE) materials, e.g., SARANEX (Dow Chemical, Midland, Mich.). The backing layer may be a monolithic or a multilaminate layer. In preferred embodiments, the backing layer is a multilaminate layer including nonlinear LDPE layer/linear LDPE layer/nonlinear LDPE layer. The backing layer can have a thickness of about 0.012 mm (0.5 mil) to 0.125 mm (5 mil); preferably about 0.025 mm (1 mil) to 0.1 mm (4 mil); more preferably about 0.0625 mm (1.5 mil) to 0.0875 mm (3.5 mil).

A wide variety of materials that can be used for fabricating the various layers of the transdermal delivery patches according to this invention have been described above. It is contemplated that the use of materials other than those specifically disclosed herein, including those which may hereafter become known to the art to be capable of performing the necessary functions is practicable.

Transdermal flux can be measured with a standard procedure using Franz cells or using an array of formulations. Flux experiments were done on isolated human cadaver epidermis. With Franz cells, in each Franz diffusion cell a disc of epidermis is placed on the receptor compartment. A transdermal delivery system is placed over the diffusion area (1.98 cm²) in the center of the receptor. The donor compartment is then added and clamped to the assembly. At time 0, receptor solution (between 21 and 24 ml, exactly measured) is added into the receptor compartment and the cell maintained at 35° C. This temperature yields a skin surface temperature of 30-32° C. Samples of the receptor compartment are taken periodically to determine the skin flux and analyzed by HPLC. In testing flux with an array of transdermal miniature patches, formulations are prepared by mixing stock solutions of each of the mixture components of formulation in organic solvents (typically 15 wt % solids), followed by a mixing process. The mixtures are then aliquoted onto arrays as 4-mm diameter drops and allowed to dry, leaving behind solid samples or “dots.” (i.e., mini-patches). The array of miniature patches is then tested individually for skin flux using a permeation array, whose principle is similar to that of an array of miniature Franz cells. The test array has a plurality of cells, a piece of isolated human epidermis large enough to cover the whole array, and a multiple well plate with wells acting as the receptor compartments filled with receptor medium. The assembled permeation arrays are stored at 32° C. and 60% relative humidity for the duration of the permeation experiments. Receptor fluid is auto-sampled from each of the permeation wells at regular intervals and then measured by HPLC for flux of the drug.

Methods of Manufacture

The transdermal devices are manufactured according to known methodology. For example, in an embodiment, a solution of the polymeric reservoir material, as described above, is added to a double planetary mixer, followed by addition of desired amounts of the drug, permeation enhancers, and other ingredients that may be needed. Preferably, the polymeric reservoir material is an acrylate material. The acrylate material is solubilized in an organic solvent, e.g., ethanol, ethyl acetate, hexane, and the like. The mixer is then closed and activated for a period of time to achieve acceptable uniformity of the ingredients. The mixer is attached by means of connectors to a suitable casting die located at one end of a casting/film drying line. The mixer is pressurized using nitrogen to feed solution to the casting die. Solution is cast as a wet film onto a moving siliconized polyester web. The web is drawn through the lines and a series of ovens are used to evaporate the casting solvent to acceptable residual limits. The dried reservoir film is then laminated to a selected backing membrane and the laminate is wound onto the take-up rolls. In subsequent operations, individual transdermal patches are die-cut, separated and unit-packaged using suitable pouchstock. Patches are placed in cartons using conventional equipment. In another process, the drug reservoir can be formed using dry-blending and thermal film-forming using equipment known in the art. Preferably, the materials are dry blended and extruded using a slot die followed by calendering to an appropriate thickness.

Such patches can be applied to the body surface of a patient. When a prolonged therapeutic effect is desired, after the prescribed time, the used patch is removed and a fresh system applied to a new location. In such cases, blood levels will remain close to constant

### EXAMPLES

Below are examples of specific embodiments for carrying out the present invention. The examples are offered for illustrative purposes only, and are not intended to limit the scope of the present invention in any way. In the following examples all percentages are by weight unless noted otherwise. T_{g} was determined by DSC (Differential Scanning Calorimetry) with 10° C./min heating rate. Modulus G′ was storage modulus at 25° C. and 1 rad/s frequency (Frequency sweep experiment was conducted using AR-2000 rheometer from TA Instruments (TA Instruments, 109 Lukens Drive, New Castle, Del. 19720). The test conditions were: strain 1%, temperature 25° C., frequency range 0.1 to 100 rad/s, gap around 1000 micron). Creep compliance tests were conducted using AR-2000 rheometer from TA Instruments. The test conditions were: stress 1000 dyn/cm², temperature 30° C., time 3600 seconds, gap around 1000 microns. One skilled in the art will know how to measure T_{g}, creep compliance, and storage modulus in view of the present disclosure. DURO-TAK® adhesives such as DURO-TAK® 87-4287 are available from National Starch & Chemicals, Bridgewater, N.J. in 2005 and at the time of the filing of the present application and their chemical and physical properties are assessable by those skilled in the art.

### Example 1

A monomer mix containing butyl acrylate, 2-hydroxyethyl acrylate, t-octyl acrylamide, acrylic acid, ethyl acetate (solvent), and 2,2′-azobisisobutyronitrile (AIBN) (polymerization initiator) was prepared. A fraction was charged to an appropriate vessel and heated to reflux with stirring. The remainder was added to the vessel over time. The ratios of the monomers and initiator added totally, i.e., butyl acrylate:2-hydroxyethyl acrylate:t-octyl acrylamide:acrylic acid:AIBN were 59:25.5:9.5:6:2. The material was then held at reflux for a suitable period of time. At the end of the hold period, the contents were cooled to room temperature and the solution polymer discharged. The dry film made from this polyacrylate formulation had storage modulus of around 9×10⁵ dyn/cm², creep compliance of around 7×10⁻⁵ cm²/dyn, and glass transition temperature of −8° C., and consequently was too stiff to provide adequate adhesive properties alone. This formed a proadhesive.

### Example 2

A monomer mix containing butyl acrylate, 2-hydroxypropyl acrylate, t-octyl acrylamide, acrylic acid, ethyl acetate (solvent), and 2,2′-azobisisobutyronitrile (AIBN) (polymerization initiator) was prepared. A fraction was charged to an appropriate vessel and heated to reflux with stirring. The remainder was added to the vessel over time. The material was held at reflux for a suitable period of time. The ratios of the monomers and initiator added totally, i.e., butyl acrylate:2-hydroxypropyl acrylate:t-octyl acrylamide:acrylic acid:AIBN were 59:25.5:9.5:6:2. At the end of the hold period, the contents were cooled to room temperature and the solution polymer discharged. The dry film made from this polyacrylate formulation had storage modulus of around 8×10⁵ dyn/cm², creep compliance of around 4×10⁻⁵ cm²/dyn, and glass transition temperature of −8° C., and consequently was too stiff to provide adequate adhesive properties alone. This formed a proadhesive.

### Example 3

A monomer mix containing vinyl acetate, 2-hydroxyethyl acrylate, 2-ethylhexyl acrylate, ethyl acetate (solvent), and 2,2′-azobisisobutyronitrile (AIBN) (polymerization initiator) was prepared. A fraction was charged to an appropriate vessel and heated to reflux with stirring. The remainder was added to the vessel over time. The material was held at reflux for a suitable period of time. The ratios of the monomers and initiator added totally, i.e., vinyl acetate:2-hydroxyethyl acrylate:2-ethylhexyl acrylate:AIBN were 50:10:40:1.2. At the end of the hold period, the contents were cooled to room temperature and the solution polymer discharged. The dry film made from this polyacrylate formulation had storage modulus of around 2×10⁶ dyn/cm², creep compliance of around 4×10⁻⁶ cm²/dyn, and glass transition temperature of −14° C., and consequently was too stiff to provide adequate adhesive properties alone. This formed a proadhesive.

### Example 4

A monomer mix containing vinyl acetate, 2-hydroxyethyl acrylate, 2-ethylhexyl acrylate, ethyl acetate (solvent), and 2,2′-azobisisobutyronitrile (AIBN) (polymerization initiator) was prepared. A fraction was charged to an appropriate vessel and heated to reflux with stirring. The remainder was added to the vessel over time. The ratios of the monomers and initiator added totally, i.e., vinyl acetate:2-hydroxyethyl acrylate:2-ethylhexyl acrylate:AIBN were 60:20:20:1.2. The material was held at reflux for a suitable period of time. At the end of the hold period, the contents were cooled to room temperature and the solution polymer discharged. The dry film made from this polyacrylate formulation had storage modulus of around 4×10⁶dyn/cm², creep compliance of around 2×10⁻⁶ cm²/dyn, and glass transition temperature of −8° C., and consequently was too stiff to provide adequate adhesive properties alone. This formed a proadhesive.

The polyacrylates of Examples 1 to 4 can be used to make a risperidone reservoir for a transdermal delivery system of the present invention.

### Example 5

Polyacrylate adhesive DURO-TAK® 87-2287 (from National Starch & Chemical Co.) and proadhesives from EXAMPLE 3 and EXAMPLE 4 were analyzed with and without permeation enhancers. The data in Table 1 clearly demonstrate the effect of enhancer on the properties of current commercial acrylic adhesive as well as the novel polyacrylate compositions described in this application. DURO-TAK® 87-2287 adhesive with a T_{g} of −34° C. had severe cold flow at 20% lauryl lactate (LL) loading level. Such cold flow phenomenon is the reason this adhesive and most similar commercial pressure sensitive adhesive systems are not suitable for applications where relatively high loadings of enhancers are needed. DURO-TAK® 87-2287 had unacceptable rheological properties (severe cold flow) for transdermal application in the presence of 20% lauryl lactate. (Based on this invention, it was also found that many other PSA's with T_{g}, creep compliance and storage modulus similar to DURO-TAK® 87-2287 in the range suitable for PSA “as is” would behave similarly). The data in Table 1 demonstrated that the current commercial acrylate PSA were not suitable for applications where high loading of enhancers is needed. It was found that transdermal patches started to have undesirable Theological properties, such as the tendency to cold flow and low cohesive strength, when creep compliance is larger than 6×10⁻⁴ cm²/dyn. It has been found that typically for the prior commercial transdermal PSAs, enhancer loading is usually less than 20% due to the impact of enhancer on PSA Theological properties.

The improvement of enhancer tolerance using the novel polyacrylate composition described in this application can also be seen from the data in Table 1. By increasing the ratio of hard to soft monomer in the formulation, the glass transition temperatures were increased. The molecular weight was also increased. As a result, the polyacrylate compositions described in EXAMPLES 3 and 4 have higher modulus and lower creep compliance as can be seen from the data in Table 1. This resulted in polyacrylate compositions not suitable for pressure sensitive adhesive application in pure form due to high modulus. However, these polyacrylate compositions have better enhancer tolerance. As a result, the compositions after the addition of 35 wt % LL have the desired Theological properties for transdermal application. As can be seen from the data in Table 1, desirable creep compliance was still present when enhancer loading was 35 wt %. Further adding risperidone to result in a therapeutic dose for treating neurological disorders such as schizophrenia and bipolar disorder is expected to result in a composition having acceptable modulus G′ and creep compliance. Also, the proadhesives used for making the transdermal patches of the present invention are made to provide the capability to incorporate a large amount of permeation enhancers (and drugs such as risperidone). Table 1 is an illustration that permeation enhancers can be dissolved in the proadhesive to result in an adhesive with acceptable rheological property such as that described above. It is expected that the proadhesives will be able to hold a large amount of permeation enhancers such as lauric acid, ester of lauric acid, oleic acid, ester of oleic acid, laureth-2, ester of laureth-2, lactic acid, ester of lactic acid, pyroglutamate, and n-lauroyl sarcosine, glyceryl monolaurate, glyceryl monooleate, myristyl lactate. Such permeation enhancers can be used to aid the transdermal flux of risperidone.

TABLE 1
Effect of enhancer lauryl lactate on adhesive properties.

| | | | Creep |
|---|---|---|---|
| | | Modulus G′, | compliance, |
| Sample | T_{g}, ° C. | dyn/cm² | cm²/dyn |
| DURO-TAK ® 87-2287 | −34 | 2.1 × 10⁵ | 1.3 × 10⁻⁴ |
| Polyacrylate composition from | −14 | 2.0 × 10⁶ | 4.0 × 10⁻⁶ |
| EXAMPLE 3 | | | |
| Polyacrylate composition from | −8 | 4.0 × 10⁶ | 2.0 × 10⁻⁶ |
| EXAMPLE 4 | | | |
| 20 wt % LL in DURO-TAK ® | — | 5.6 × 10⁴ | 1.84 × 10⁻³ |
| 87-2287 | | | |
| 34 wt % LL in Polyacrylate | — | 1.0 × 10⁵ | 3.2 × 10⁻⁴ |
| composition from EXAMPLE 3 | | | |
| 35 wt % LL in Polyacrylate | — | 1.2 × 10⁵ | 4.0 × 10⁻⁴ |
| composition from EXAMPLE 4 | | | |

### Experiments with Risperidone

### Example A

Several formulations were tested using a high throughput skin flux platform.

The formulations were prepared and evaluated for flux through isolated human epidermis. Formulations were prepared by mixing stock solutions of each of the mixture components in organic solvents (typically 15wt % solid content in ethyl acetate, methanol and/or ethanol), followed by a mixing process. The mixtures were then aliquoted onto 16×24 arrays as 4-mm diameter drops and allowed to dry. The resulting 384 miniature patches were then tested in parallel for skin flux using a 384-well permeation array. Each permeation array consisted of the 384 miniature patch array, a piece of isolated human epidermis large enough to cover the whole array, and a 384-well plate acting as the receptor compartment and which was filled with receptor medium. The assembled permeation arrays were stored at 32° C. and 60% relative humidity for the duration of the permeation experiments. Receptor fluid was auto-sampled from each of the permeation wells at regular intervals and then measured by High performance liquid chromatrography for risperidone content in order to determine the flux profile and measure the flux at steady state. Every formulation was replicated at least 3 times in order to ensure accuracy. The formulations could also have been tested on conventional Franz cells, which is a standard tool for one skilled in the art of transdermal formulation development and results would have been similar.

The fluxes determined using the method described above are presented in Table 2, which shows the mean fluxes over a period (0-54 hr) for a number of risperidone transdermal formulations in two acrylate adhesives. DURO-TAK® 87-900A adhesive (available from National Starch Corporation) is a commercial polyacrylate adhesive with no functional monomer and no vinyl acetate present in the structure. DURO-TAK® 87-900A adhesive is made from mostly 2-ethylhexyl acrylate, butylacrylate, methyl methacrylate, and tertiary-octyl acrylamide. In one aspect of the present invention, one type of useful acrylate polymer for making a risperidone transdermal delivery patch is one that comprises, and preferably consists of 2-hydroxyethyl acrylate, vinyl acetate and 2-ethylhexyl acrylate. An example is DURO-TAK® 87-4287 polyacrylate adhesive (available from National Starch & Chemical Co.), which is a terpolymer having a monomer composition of 2-6wt % 2-hydroxyethyl acrylate, with the rest being vinyl acetate (20-40 wt %)and 2-ethylhexyl acrylate (55-75 wt %). DURO-TAK® 87-4287 acrylate polymer has a T_{g} of −38C, storage modulus of 3.6×10⁵dyn/cm² and creep compliance of about 5×10⁻⁵ cm²/dyn. Risperidone loadings in the adhesive matrix were about 6 wt %. Some of the formulations were examples of formulations were examples of matrix formulations that gave the desired flux range of 2.1-5.4 μg/cm²-hr. Table 3 shows the rheological properties for risperidone transdermal formulations listed in Table 2. As expected, modulus decreased and creep compliance increased as the addition of enhancers soften the adhesive matrix.

TABLE 2
Mean Fluxes for Various Risperidone Transdermal Formulations

| | | | Mean Flux |
|---|---|---|---|
| Example # | Adhesive # | Enhancer(s): (wt %) | (μg/cm²-hr) |
| a | DURO-TAK ® 87-900A | none (control) | 1 |
| b | DURO-TAK ® 87-900A | LRA (2) | 1.7 |
| c | DURO-TAK ® 87-900A | OA (6), NLS (1) | 2.1 |
| d | DURO-TAK ® 87-4287 | OA (6), NLS (1) | 2.1 |
| e | DURO-TAK ® 87-4287 | LL (22), LRA (2) | 5.4 |
| f | DURO-TAK ® 87-4287 | LL (18), LRA (2) | 4.1 |

TABLE 3
Rheological properties for Various Risperidone
Transdermal Formulations

| | | | | creep |
|---|---|---|---|---|
| Example | | Enhancer(s): | Modulus | compliance |
| # | Adhesive # | (wt %) | (dyn/cm²) | (cm²/dyn) |
| a | DURO-TAK ® | none (control) | 6.1 × 10⁵ | 7.1 × 10⁻⁵ |
| | 87-900A | | | |
| b | DURO-TAK ® | LRA (2) | 6.0 × 10⁵ | 8.7 × 10⁻⁵ |
| | 87-900A | | | |
| c | DURO-TAK ® | OA (6), NLS (1) | 5.0 × 10⁵ | 1.3 × 10⁻⁴ |
| | 87-900A | | | |
| d | DURO-TAK ® | OA (6), NLS (1) | 2.7 × 10⁵ | 6.2 × 10⁻⁵ |
| | 87-4287 | | | |
| e | DURO-TAK ® | LL (22), LRA (2) | 6.7 × 10³ | 4.4 × 10⁻⁴ |
| | 87-4287 | | | |
| f | DURO-TAK ® | LL (18), LRA (2) | 9.1 × 10³ | 3.3 × 10⁻⁴ |
| | 87-4287 | | | |

### Example B

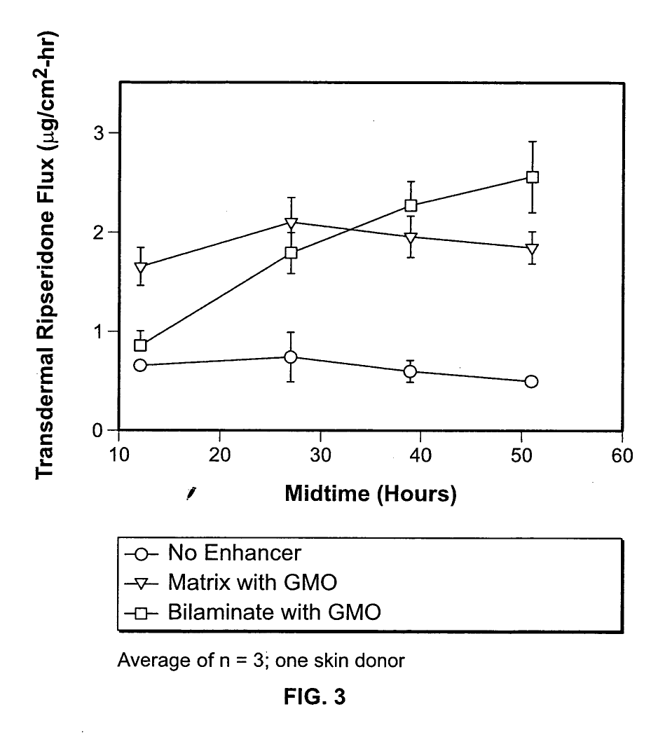

Transdermal risperidone delivery systems were made and tested for flux. FIG. 3 is a graph that shows an in vitro transdermal flux comparison of an example of a bilaminate construction to an embodiment of a matrix with risperidone delivery. The data were averaged over three runs and all experiments were done on skin from the same donor. Flux experiments were performed using a procedure similar to Example A. The curve with the circular data points (circle ∘) is from a formulation without enhancer. The curve with the triangular data points (inverted Δ) is from a formulation with GMO. The curve with the square data points (square □) is from a formulation on a bilaminate with GMO. These systems were made by incorporating drug risperidone (7 wt %) plus enhancers (GMO, octyl pyrrrolidone) with National Starch DURO-TAK® 87-4287 adhesive in a small vial with solvents. The solution was cast on the peelable liner comprised of siliconized polyester and allowed to dry. The final thickness was about 5 mils (0.125 mm), and final enhancer concentrations were 6% GMO and 25% octyl pyrrolidone. FIG. 3 shows that both the matrix device and the bilaminate device with GMO provided acceptable flux of risperidone.

### Example C

Transdermal risperidone delivery systems are made using drug and enhancer tolerant polyacrylates of increased polarity. These proadhesive are expected to be capable of dissolving more risperidone and enhancer(s). Using the same method as described in Example A above, formulation with 20 wt % risperidone, 25 wt % lauryl lactate, 5 wt % lauryl acid, and 50 wt % polyacrylate are prepared and evaluated for flux through isolated human epidermis. The polyacrylate is the polyacrylate of Example 3. This polyacrylate is a copolymer and consisted of 50 wt % vinyl acetate, 10 wt % 2-hydroxyethyl acrylate, and 40 wt % 2-ethylhexyl acrylate. Such systems are expected to be still mono-phasic and result in transdermal flux values of around 2 μg /cm²-hr or higher, possibly 4 μg/cm²-hr or higher, possibly 10 μg/cm²-hr or higher.

### Example D

Transdermal risperidone delivery systems are made using drug and enhancer tolerant polyacrylates of increased polarity. These proadhesive are expected to be capable of dissolving more risperidone and enhancer(s). Using the same method as described in Example A above, formulation with 20 wt % risperidone, 20 wt % oleic acid (OA), 5 wt % N-lauryl sarcosine (NLS), and 55 wt % polyacrylate are prepared and evaluated for flux through isolated human epidermis. The polyacrylate is the polyacrylate of Example 1. The polyacrylate of Example 1 is a copolymer and consisted of 59 wt % butyl acrylate, 25.5 wt % 2-hydroxyethyl acrylate, 9.5wt % t-octyl acrylamide, and 6 wt % acrylic acid. Such systems are expected to be still mono-phasic and result in transdermal flux values of around 2 μg/cm²-hr or higher, possibly 4 μg/cm²-hr or higher, possibly 10 μg/cm²-hr or higher.

### Example E

Transdermal risperidone delivery systems are made using drug and enhancer tolerant polyacrylates of increased polarity. These proadhesive are expected to be capable of dissolving more risperidone and enhancer(s). Using the same method as described in Example B above, formulation with 20 wt % risperidone, 20 wt % oleic acid (OA), 5 wt % N-lauryl sarcosine (NLS), and 55 wt % polyacrylate are prepared and evaluated for flux through isolated human epidermis. The polyacrylate is the polyacrylate of Example 2. The polyacrylate of Example 2 is a copolymer and consisted of 59 wt % butyl acrylate, 25.5 wt % 2-hydroxypropyl acrylate, 9.5wt % t-octyl acrylamide, and 6 wt % acrylic acid. Such systems are expected to be still mono-phasic and result in transdermal flux values of around 2 μg/cm²-hr or higher, possibly 4 μg/cm²-hr or higher, possibly 10 μg/cm²-hr or higher.

### Example F

Transdermal risperidone delivery systems are made using drug and enhancer tolerant polyacrylates of increased polarity. These proadhesive are expected to be capable of dissolving more risperidone and enhancer(s). Using the same method as described in Example A above, formulation with 10 wt % risperidone, 25 wt % lauryl lactate, 5 wt % lauryl acid, and 60 wt % polyacrylate are prepared and evaluated for flux through isolated human epidermis. The polyacrylate is the polyacrylate of Example 3. This polyacrylate is a copolymer and consisted of 50 wt % vinyl acetate, 10 wt % 2-hydroxyethyl acrylate, and 40 wt % 2-ethylhexyl acrylate. Such systems are expected to be still mono-phasic and result in transdermal flux values of around 2 μg/cm²-hr or higher, possibly 4 μg/cm 2-hr or higher, possibly 10 μg/cm²-hr or higher.

### Example G

Transdermal risperidone delivery systems are made using drug and enhancer tolerant polyacrylates of increased polarity. These proadhesive are expected to be capable of dissolving more risperidone and enhancer(s). Using the same method as described in Example A above, formulation with 10 wt % risperidone, 30 wt % lauryl lactate, 5 wt % lauryl acid, and 55 wt % polyacrylate are prepared and evaluated for flux through isolated human epidermis. The polyacrylate is the polyacrylate of Example 4. The polyacrylate of Example 4 is a copolymer and consisted of 60 wt % vinyl acetate, 20 wt % 2-hydroxyethyl acrylate, and 20 wt % 2-ethylhexyl acrylate. Such systems are expected to be still mono-phasic and result in transdermal flux values of around 2 μg/cm²-hr or higher, possibly 4 μg/cm²-hr or higher, possibly 10 μg/cm²-hr or higher.

The entire disclosure of each patent, patent application, and publication cited or described in this document is hereby incorporated herein by reference. The practice of the present invention will employ, unless otherwise indicated, conventional methods used by those in pharmaceutical product development within those of skill of the art. Embodiments of the present invention have been described with specificity. The embodiments are intended to be illustrative in all respects, rather than restrictive, of the present invention. It is to be understood that various combinations and permutations of various constituents, parts and components of the schemes disclosed herein can be implemented by one skilled in the art without departing from the scope of the present invention.

What is claimed is:

1. A method of making a drug reservoir for transdermal risperidone delivery, comprising: providing a solution of an acrylate polymer having polar functionality, dissolving risperidone and permeation enhancer in the solution, drying the solution to form a drug reservoir with 6 wt % or more of risperidone dissolved in the drug reservoir such that the drug reservoir can deliver the risperidone at a flux of greater than 2 mg per day at greater than 2 μg/cm²-hr, the polymer constitutes 40 wt % to 90 wt % in solids of the drug reservoir, the drug reservoir being applicable as a pressure sensitive adhesive to a body surface.

2. The method of claim 1 wherein the drug reservoir is a multiple day use reservoir and the flux is greater than 4 μg/cm²-hr.

3. The method of claim 2 comprising dissolving more than 15 wt % risperidone and dissolving permeation enhancer in the solution such that the risperidone and permeation enhancer make up greater than 30 wt % dissolved solids in the drug reservoir.

4. The method of claim 2 wherein the acrylate polymer has functional monomer, constitutes 45 wt % to 80 wt % of the reservoir and has dissolved therein at least 30 wt % for the risperidone and permeation enhancer combination, the acrylate polymer having a T_{g} of greater than −15° C. if without permeation enhancer and without drug.

5. The method of claim 4 wherein the acrylate polymer has no more than 60 wt % soft monomer component, has at least 40 wt % hard monomer component at least a portion of which being functional monomer, and 1 to 35 wt % functional monomer component.

6. The method of claim 4 wherein the reservoir has a glass transition temperature T_{g} of less than −10° C. whereas the acrylate polymer has a T_{g} of greater than −15° C. and a creep compliance of 6×10⁻⁵ cm²/dyn to 2×10⁻⁶ cm²/dyn.

7. The method of claim 4 wherein the acrylate polymer includes (i) 40 to 50 wt % of soft alkyl acrylate monomer component, in which each soft alkyl acrylate monomer having a homopolymer T_{g} of −80 to −20° C., (ii) 10 to 60 wt % of nonfunctional hard modifying monomer component, in which each hard modifying monomer having a homopolymer T_{g} of 0 to 250° C., and (iii) up to 30% by weight of functional monomer component, wherein each soft monomer is an alkyl acrylate monomer having 4 to 10 carbon atoms in the alkyl group.

8. The method of claim 4 wherein the acrylate polymer includes a soft acrylate monomer selected from the group consisting of butyl, hexyl, 2-ethylhexyl, octyl, and dodecyl acrylates and isomers thereof.

9. The method of claim 4 wherein the acrylate polymer includes 40 to 50 wt % of a soft alkyl acrylate monomer that has a homopolymer T_{g} of less than −20° C.

10. The method of claim 4 wherein the acrylate polymer has a T_{g} of 0 to −20° C. if without drug and permeation enhancer, and the reservoir having the dissolved drug and permeation enhancer has a T_{g} of −10 to −20° C., a creep compliance of 1×10⁴ cm²/dyn to 6×10⁻⁴ cm²/dyn and storage modulus of 1×10⁵ dyn/cm² to 8×10⁵ dyn/cm².

11. The method of claim 4 comprising incorporating permeation enhancer and risperidone in the acrylate polymer in single phase, wherein the acrylate polymer has a T_{g} of 0 to −20° C., storage modulus of 8×10⁵ dyn/cm² or above if without drug and permeation enhancer, and the reservoir with drug and permeation enhancer has a T_{g} of −10 to −20° C., a creep compliance of 1×10−4 cm²/dyn to 6×10⁻⁴ cm²/dyn and storage modulus of 1×10⁵ dyn/cm² to 8×10⁵ dyn/cm².

12. The method of claim 4 comprising providing the acrylate polymer having monomer components of 50 to 60 wt % vinyl acetate, 10-20 wt % hydroxyethyl acrylate, and 20-40 wt % 2-ethylhexyl acrylate.

13. The method of claim 4 comprising providing the acrylate polymer having monomer components of 55 to 65 wt % butyl acrylate, 5 to 15 wt % t-octyl acrylamide, 20 to 30 wt % hydroxyethyl or hydroxypropyl acrylate and 5 to 10 wt % acid monomer.

14. A method of making a transdermal risperidone drug delivery reservoir, comprising: providing for a reservoir a polyacrylate proadhesive containing function group and having a T_{g} of greater than −15° C., creep compliance of 6×10⁻⁵ cm²/dyn to 2×10⁻⁶ cm²/dyn, and storage modulus of 8×10⁵ dyn/cm² or above, dissolving risperidone and permeation enhancer in the proadhesive with a concentration of greater than 30 wt % solids of drug and permeation enhancer combination such that the resulting reservoir is applicable as a pressure sensitive adhesive for transdermal drug delivery, the resulting reservoir having a T_{g} of −10 to −30° C., a creep compliance of 1×10−4cm²/dyn to 6×10⁻⁴ cm²/dyn and storage modulus of 1×10⁵dyn/cm² to 8×10⁵ dyn/cm².

15. A device for transdermal administration of risperidone to an individual in need thereof for therapy through a body surface, comprising a backing and a drug reservoir comprising acrylate polymer having polar functional group, dissolved risperidone of 6 wt % or more on solids, permeation enhancer of sufficient amount to deliver the risperidone at a flux of greater than 2 mg per day through a body surface.

16. The device of claim 15 wherein the flux is greater than 4 μg/cm²-hr transdermally.

17. The device of claim 15 wherein the drug reservoir has 15 wt % or more of risperidone and greater than 30 wt % of risperidone together with permeation enhancer.

18. The device of claim 17 wherein the drug reservoir has at least 30 wt % solids of risperidone with permeation enhancer together and the acrylate polymer comprises 40 wt % to 90 wt % of the drug reservoir, wherein the drug reservoir maintains appropriate pressure sensitive adhesive properties applicable to the body surface.

19. The device of claim 15 wherein the acrylate polymer has no more than 60 wt % soft monomer, at least 40 wt % hard monomer component at least a portion of which is also functional monomer and 1 to 35 wt % functional monomer component, the acrylate polymer constituting 45 wt % to 80 wt % of the reservoir and having a solubility of at least 30 wt % for the risperidone and permeation enhancer combination, the acrylate polymer having a T_{g} of greater than −15° C. if without permeation enhancer and without drug, the reservoir having pressure sensitive adhesive properties applicable to the body surface for transdermal delivery.

20. The device of claim 15 wherein the reservoir in the device includes permeation enhancer wherein the reservoir is of a composition having a creep compliance of 6×10⁻⁵ cm²/dyn to 2×10⁻⁶ cm²/dyn if the reservoir is without drug and without permeation enhancer.

21. The device of claim 15 wherein the acrylate polymer includes 5 to 35 wt % functional monomer.

22. The device of claim 15 wherein the acrylate polymer includes an acrylic copolymer resulting from (i) 40 to 50 wt % of soft alkyl acrylate monomer component, in which each soft alkyl acrylate monomer having a homopolymer T_{g} of −80 to −20° C., (ii) 10 to 60 wt % of nonfunctional hard modifying monomer component, in which each hard modifying monomer having a homopolymer T_{g} of 0 to 250° C., and (iii) functional monomer component of up to 35 wt %.

23. The device of claim 15 wherein the acrylate polymer has (i) 40 to 50 wt % of soft alkyl acrylate monomer component, in which each soft alkyl acrylate monomer having a homopolymer T_{g} of −80 to −20° C., (ii) 40 to 60 wt % of nonfunctional hard modifying monomer component, in which each hard modifying monomer having a homopolymer T_{g} of 0 to 250° C., and (iii) one or more functional monomers of up to 35 wt %, wherein the soft monomer is an alkyl acrylate monomer having 4 to 10 carbon atoms in the alkyl group.

24. The device of claim 15 wherein the acrylate polymer includes a soft acrylate monomer selected from the group consisting of butyl, hexyl, 2-ethylhexyl, octyl, and dodecyl acrylates and isomers thereof.

25. The device of claim 15 wherein the acrylate polymer includes 40 to 50 wt % of a soft alkyl acrylate monomer having a homopolymer T_{g} of less than −20° C.

26. The device of claim 15 wherein the acrylate polymer includes 40 to 50 wt % of a soft alkyl acrylate monomer having a homopolymer T_{g} of less than −20° C., hard modifying monomer having a homopolymer T_{g} of higher than 20° C., and functional monomer having acidic group.

27. The device of claim 15 wherein the acrylate polymer includes hard modifying monomer having a homopolymer T_{g} of 0 to 250° C., wherein the permeation enhancer and the risperidone are dissolved in the acrylate polymer and the acrylate polymer has a T_{g} of 0 to −20° C. and a creep compliance of 6×10⁻⁵ cm²/dyn to 2×10⁻⁶ cm²/dyn without the risperidone and permeation enhancer dissolved therein, whereas the reservoir with the dissolved drug and permeation enhancer has a creep compliance of less than 1×10⁻³ cm²/dyn and storage modulus of 1×10⁵ dyn/cm² to 8×10⁵ dyn/cm.

28. The device of claim 15 wherein the acrylate polymer includes hard modifying monomer having a homopolymer T_{g} of 40 to 100° C.

29. The device of claim 15 wherein the acrylate polymer includes hard modifying monomer selected from the group consisting of vinyl acetate, methyl acrylate, and methyl methacrylate.

30. The device of claim 15 wherein the acrylate polymer has acidic group and hydroxyl group therein and includes 5 to 15 wt % nonfunctional hard monomer.

31. The device of claim 15 wherein the acrylate polymer includes monomer components of 50 to 60 wt % vinyl acetate, 10-20 wt % hydroxyethyl acrylate, and 20-40 wt % 2-ethylhexyl acrylate.

32. The device of claim 15 wherein the acrylate polymer includes functional monomer selected from the group consisting of acrylic acid, hydroxyethyl acrylate, and hydroxypropyl acrylate.

33. The device of claim 15 wherein the permeation enhancer and the risperidone are dissolved in the acrylate polymer and the acrylate polymer has a T_{g} of 0 to −20° C., a creep compliance of 6×10⁻⁵ cm²/dyn to 2×10⁻⁶ cm²/dyn without the risperidone and permeation enhancer, whereas with the dissolved risperidone and permeation enhancer the acrylate polymer forms a reservoir with a T_{g} of −10 to −20° C., a creep compliance of less than 1×10⁻³ cm²/dyn and storage modulus of 1×10⁵dyn/cm² to 8×10⁵ dyn/cm².

34. The device of claim 15 wherein the acrylate polymer has a T_{g} of 0 to −20° C. if without drug and permeation enhancer, whereas the acrylate polymer with drug and permeation enhancer at above 30 wt % in a single phase forms a reservoir with a T_{g} of −10 to −20° C., a creep compliance of 1×10⁻⁴ cm²/dyn to 6×10⁻⁴ cm²/dyn and storage modulus of 1×10⁵dyn/cm² to 8×10⁵ dyn/cm².

35. The device of claim 15 wherein the acrylate polymer has a T_{g} of 0 to −20° C., storage modulus of 8×10⁵ dyn/cm² or above ° C. if without drug and permeation enhancer, whereas the acrylate polymer with drug and permeation enhancer at above 30 wt % forms a reservoir with a T_{g} of −10 to −40° C., a creep compliance of 1×10⁻⁴ cm²/dyn to 6×10⁻⁴ cm²/dyn and storage modulus of 1×10⁵dyn/cm² to 8×10⁵ dyn/cm².

36. The device of claim 15 having a permeation enhancer selected from the group consisting of lauric acid, ester of lauric acid, oleic acid, ester of oleic acid, laureth-2, ester of laureth-2, lactic acid, ester of lactic acid, pyroglutamate, and n-lauroyl sarcosine, glyceryl monolaurate, glyceryl monooleate, myristyl lactate.

37. The device of claim 15 wherein the drug reservoir includes pyroglutamate or acetic acid as a permeation enhancer.

38. The device of claim 15 wherein the drug reservoir includes acetic acid as a permeation enhancer and has greater than 10 wt % of risperidone.

39. The device of claim 15 wherein the drug reservoir includes a permeation enhancer with acid moiety.

40. The device of claim 15 wherein the reservoir has more than 5 wt % risperidone and 20 wt % permeation enhancer content.

41. The device of claim 15 wherein the device can deliver 2 to 6 mg risperidone per day and the area of the device contacting the skin is 50 cm² or less.

42. The device of claim 15 wherein the reservoir includes pyroglutamate or acetic acid.

43. The device of claim 15 wherein the reservoir includes acetic acid and greater than 10 wt % of risperidone.

44. The device of claim 15 wherein the reservoir includes as permeation enhancer at least one of N-lauroyl sarcosine, lauryl lactate, oleic acid, and lauric acid.

45. The device of claim 15 wherein the reservoir includes as permeation enhancer at least one of N-lauroyl sarcosine, and lauric acid.
